(19) United States
(12) Patent Application Publication
(10) Pub. No.: US 2022/0106570 A1
(43) Pub. Date: Apr. 7, 2022

(54) DISEASE MODEL

(71) Applicant: NAGASAKI UNIVERSITY, Nagasaki (JP)

(72) Inventors: Tomoshi Tsuchiya, Nagasaki (JP); Takeshi Nagayasu, Nagasaki (JP); Satoshi Mizoguchi, Nagasaki (JP)

(21) Appl. No.: 17/426,899

(22) PCT Filed: Jan. 29, 2020
(86) PCT No.: PCT/JP2020/003159

§ 371 Date: Jul. 29, 2021

(30) Foreign Application Priority Data
Jan. 30, 2019 (JP) 2019-014778

(51) Int. Cl. C12N 5/071; G01N 33/50

(52) U.S. Cl. CPC C12N 5/0688; G01N 33/5088; C12N 2503/04; C12N 2502/1323; C12N 2502/28; C12N 2500/02

(57) ABSTRACT

A method for producing a disease model, including a step of introducing a cancer cell or fibroblast into a recellularized organ or tissue is provided by the present invention.

### TECHNICAL FIELD

The present invention relates to a production method of a disease model, a disease model produced by the method, and a method for screening for an agent for treating or preventing a disease by using the model.

### BACKGROUND ART

Cancer is one of the major causes of death in humans. Although research on cancer treatment methods is being actively pursued, the 5-year survival rate is still low. For example, in the case of lung cancer, the 5-year survival rate of lung cancer patients remains at about 15%. In recent years, the development of anticancer agents targeting molecules expressed in cancer has been promoted. As one of the targets, epidermal growth factor receptor (EGFR) whose overexpression is observed in various malignant tumors such as non-small cell lung cancer (NSCLC) and the like has been attracting attention. For example, it has been reported that administration of an EGFR inhibitor to NSCLC patients having an EGFR mutation that causes EGFR activation affords an antitumor effect. However, the number of patients having the gene mutation is small, and it has also been reported that cancer cells problematically have drug resistance due to a secondary mutation of the gene (e.g., non-patent document 1). Therefore, it is considered essential to understand the biological mechanism of the disease, including the mechanisms of variation and resistance acquisition, so as to provide a drug and drug combinations for treating diseases such as cancer and the like which are suitable for individual patients (e.g., non-patent document 2).

Fibrosis is a disease in which abnormal accumulation of fiber tissue is observed due to tissue damage, autoimmune response and the like. In humans, fibrosis is known to occur in various organs and tissues such as lung, liver, pancreas, kidney, heart, bone marrow, skin and the like. Fibrosis with an identifiable cause is often cured by removing the cause or administering an anti-inflammatory agent such as a steroid. On the other hand, steroids and immunosuppressants are generally used for the treatment of pulmonary fibrosis and interstitial pneumonia accompanied by fibrosis. However, as the situation stands, there is no effective treatment method that improves the prognosis, and the development of a new therapeutic agent is desired.

In the development of anticancer agents, clinical failures in the later stages of phase II and phase III clinical trials cause abandonment of the development in many cases (e.g., non-patent document 3). One of the reasons therefor is the lack of a model system that can accurately predict pharmacological effects. Cancer response to a drug is influenced by complex interactions of several factors, including tissue-specific microenvironment, mechanical stimuli, and the like. It is extremely difficult to evaluate such influences in cells conventionally cultured two-dimensionally. Therefore, the development of a model system that reproduces a disease, particularly a disease model having a three-dimensional structure, that is useful for elucidating the biological mechanism of diseases such as cancer, fibrosis and the like, and can more accurately predict a therapeutic effect on the disease is desired.

Incidentally, in the field of transplantation medicine, recellularized organs in which decellularized organ scaffolds are recellularized by autologous cells are greatly expected. Decellularized tissue scaffolds are comparatively easily obtained from animal and human tissues and organs, and therefore widely used as medical materials in clinical practice. In the field of cardiovascular surgery, porcine and bovine-derived biological valves (HANCOK II (registered trade mark), PERIMOUNT Magna (registered trade mark), human biological valve (Synegraft) (registered trade mark)) and the like are used as medical materials. In the field of orthopedic surgery, human-derived skin (AlloDerm (registered trade mark)), porcine small intestine (OASIS (registered trade mark)), artificial bone (AlloCraft C-Ring (registered trade mark)) and the like are used as medical materials. By engrafting “autologous cells” in these clinically-used medical materials, it is theoretically possible to create an autologous organ from a xenogeneic organ. As a method for engrafting autologous cells and the like, a method for decellularizing an organ and engrafting autologous cells in the organ to produce a recellularized organ has been reported (e.g., non-patent document 4). However, as far as the present inventors know, there are no reports that a disease model could be prepared from a recellularized organ.

### DOCUMENT LIST

### Non-Patent Documents

non-patent document 1: Jackman, D. M. et al., Clin. Cancer Res, 12:3908-3914 (2006)
non-patent document 2: Regales L. et al., J Clin Invest, 119(10):3000-10 (2009)
non-patent document 3: DiMasi J. A. et al., Clin Pharmacol Ther, 94(3):329-35 (2013)
non-patent document 4: Thomas H. et al., Science, 329(5991): 538-41 (2010)

### SUMMARY OF INVENTION

### Technical Problem

Therefore, the problems of the present invention are provision of a method for producing a disease model having a three-dimensional structure that can be used for elucidation of the biological mechanism of a disease and more accurate prediction of the effect of an agent for treating or preventing a disease, a method for screening for an agent for treating or preventing a disease by using a disease model produced by the method, and the like.

### Solution to Problem

The present inventors have conducted intensive studies and noted the idea that a cancer model having a three-dimensional structure and reproducing a naturally-occurring cancer can be produced by once recellularizing using normal cells and then seeding cancer cells, rather than by a method for producing a recellularized organ by seeding cancer cells in a decellularized organ, and that the effect of an anticancer agent can be predicted more accurately by using the cancer model than when using conventional cells, organs and the like. Based on this idea, they have continued research and found that an artificial lung that reflects the histopathological findings of naturally-occurring lung cancer, that is, that reproduces naturally-occurring lung cancer, can be produced when the lung is used as the organ. Based on these findings, the present inventors conducted further studies and completed the present invention.

Accordingly, the present invention provides the following.

[1] A method for producing a disease model, comprising a step of introducing a cancer cell or fibroblast into a recellularized organ or tissue.
[2] The method of [1], wherein the aforementioned organ or tissue is a lung or lung tissue.
[3] The method of [1] or [2], wherein the aforementioned cancer cell is a lung cancer cell.
[4] The method of any of [1] to [3], wherein the aforementioned cancer cells are one or more types of cells selected from the group consisting of A549 cell, PC-9 cell, H520 cell, H1975 cell, HCC827 cell and PC-6 cell.
[5] The method of any of [2] to [4], wherein the aforementioned recellularized lung or lung tissue is a lung or lung tissue produced by introducing an epithelial cell and an endothelial cell into a decellularized lung or lung tissue.
[6] A disease model produced by the method of any of [1] to [5].
[7] The disease model of [6], wherein the aforementioned disease model is a lung disease model.
[8] A method for screening for an agent for treating or preventing a disease, comprising
(1) a step of contacting the disease model of [6] or [7] with a test substance, and
(2) a step of selecting the test substance as a candidate substance for treating or preventing a disease when the contact with the test substance decreases the number of cancer cells or fibroblasts, or decreases the proliferation rate of the cell, compared with the disease model before contact with the test substance or a disease model without contact with the test substance.
   [9] The method of [8], wherein the aforementioned disease is a lung disease.
   [10] A method for evaluating a side effect of a test substance, comprising
(1) a step of contacting the disease model of [6] or [7] with the test substance, and
(2) a step of evaluating the level of damage to the disease model due to the contact with the test substance.

### Advantageous Effects of Invention

According to the present invention, a method for producing a disease model having a three-dimensional structure that reproduces a naturally occurring disease and exhibits drug responsiveness similar to that of the naturally occurring disease is provided. Using the disease model thus produced, candidate substances for an agent for treating or preventing a disease can be screened for more accurately as compared with conventional methods. For example, when the lung is used, a physiological mechanical stress such as perfusion to pulmonary blood vessels, addition of respiratory movement, or the like can be added to the lung disease model in a bioreactor. Therefore, the disease model may also be useful in elucidating the biological mechanism of a disease, including elucidation of mechanobiology. Furthermore, progression of the introduced cells, especially cancer cells, can be observed during the process of producing the above-mentioned disease model. Therefore, the disease model may also be useful in studying the progression of a disease, particularly a lung disease.

### BRIEF DESCRIPTION OF DRAWINGS

FIG. 1 shows a schematic diagram of the production method of a lung disease model and a macroscopic image of rat lung during recellularization.

FIG. 2 shows a macroscopic image of rat lung after decellularization (left Figure) and a macroscopic image of rat lung during recellularization (right Figure).

FIG. 3 shows a Hematoxylin-Eosin-staining image of the recellularized lung (also referred to as recellularized lung) of a rat after decellularization. scale bar: 100 μm

FIG. 4 shows a Hematoxylin-Eosin-staining image of the recellularized lung of a rat after recellularization. Alveolar epithelial cells and vascular endothelial cells that were perfused during recellularization engrafted in such a way as to reproduce a normal alveolar structure while maintaining the alveolar structure of the decellularized scaffold. scale bar: 200 μm, 100 μm or 50 μm

FIG. 5 shows a macroscopic image of a rat recellularized lung injected with cancer cells (PC-9 cell). A white nodule was found at the site where human lung cancer cells were injected.

FIG. 6 shows Hematoxylin-Eosin-staining images of rat recellularized lung injected with adenocarcinoma cells (A549 cells (left Figure)) or squamous carcinoma cells (H520 cells (right Figure)). Both adenocarcinoma cells and squamous carcinoma cells engrafted on the recellularized lung. scale bar: 500 μm

FIG. 7 shows Hematoxylin-Eosin-staining images of rat recellularized lung injected with adenocarcinoma cells (A549 cells (left Figure)) or squamous carcinoma cells (H520 cells (right Figure)). The cell density and morphology were different depending on the type of cancer cells. scale bar: 100 μm

FIG. 8 shows Hematoxylin-Eosin-staining images of rat recellularized lung injected with adenocarcinoma cells (A549 cells (left Figure)) or squamous carcinoma cells (H520 cells (right Figure)). A grand duct-like structure was formed, and the cells contained mucus. scale bar: 50 μm

FIG. 9 shows a Periodic Acid-Schiff staining (PAS-staining) image of rat recellularized lung injected with adenocarcinoma cells (A549 cells). Mucus colored in purplish red was found in the grand duct-like structure and the cells. scale bar: 50 μm

FIG. 10 shows a Hematoxylin-Eosin-staining image of rat recellularized lung injected with adenocarcinoma cells (A549 cells). This staining image shows progression of cancer cells from the upper right to the lower left. scale bar: 100 μm

FIG. 11 shows the results of immunostaining using an anti-MUC-1 antibody in rat recellularized lung injected with adenocarcinoma cells (A549 cells). While MUC-1 is hardly expressed in two-dimensionally cultured A549 cells (2D), the expression level increases in recellularized lung (3D). scale bar: 50 μm

FIG. 12 shows the results of immunostaining using an anti-MUC-1 antibody in rat recellularized lung injected with adenocarcinoma cells (PC-9 cells). While MUC-1 is hardly expressed in two-dimensionally cultured PC-9 cells (2D), the expression level increases in recellularized lung (3D). scale bar: 50 μm

FIG. 13 shows the results of responsiveness to an anticancer agent (gefitinib) in rat recellularized lung injected with cancer cells. When A549 cells with wild-type EGFR were used, the expression of Ki67, which is a cell proliferation marker, did not change even when gefitinib was administered. However, when EGFR mutation-positive PC-9 cells were used, administration of gefitinib decreased the positive rate of Ki67. scale bar: 50 μm

FIG. 14 shows the result of the calculation using ImageJ of the ratio of Ki67-positive cells in the rat recellularized lung used in FIG. 13. When A549 cells were used, a significant difference in the Ki67-positive cell rate was not found by the administration of gefitinib. However, when PC-9 cells were used, administration of gefitinib significantly decreased the number of Ki67 positive cells.

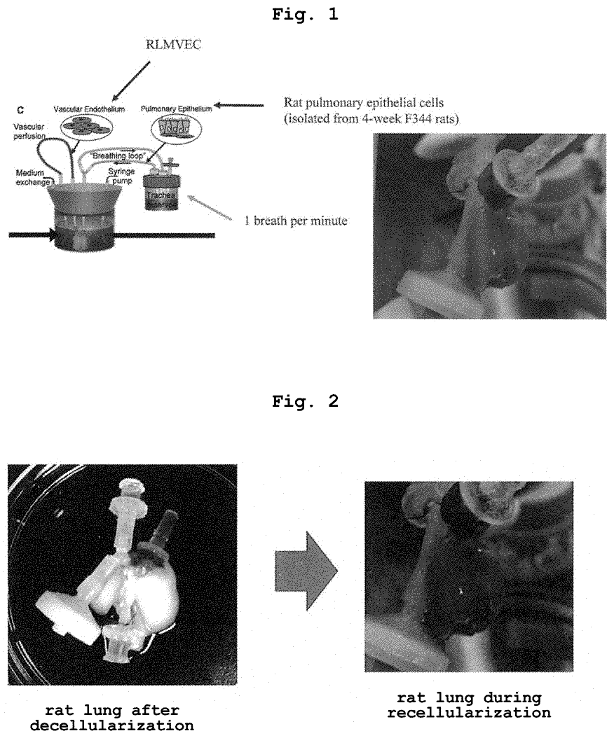

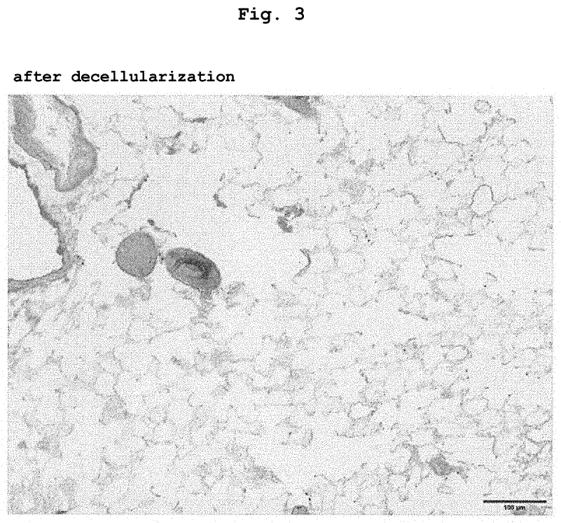

### DESCRIPTION OF EMBODIMENTS

### 1. Production Method of Disease Model

The present invention provides a method for producing a disease model, particularly a lung disease (e.g., lung cancer, lung fibrosis) model, including a step of introducing cells for reproducing a disease (hereinafter sometimes to be referred to as “cell for disease reproduction”) into a recellularized organ or tissue (hereinafter sometimes to be referred to as “the production method of the present invention”). In the present specification, the “disease model” means a recellularized organ or tissue in which cells for disease reproduction (preferably cancer cells) engrafted, and the disease model preferably reproduces a disease (e.g., cancer, fibrosis) caused by the engrafted cells.

As shown in the following Examples, the lung cancer model produced by the production method of the present invention was shown to reflect the histopathological findings of naturally-occurring lung cancer, that is, reproduce naturally-occurring lung cancer (FIGS. 7-9). To be specific, when adenocarcinoma cells were used, a nodule was found at the site where the cells were introduced, a grand duct-like structure which is a histopathologic finding of adenocarcinoma was formed, and the cells contained mucus. In addition, similar to naturally-occurring lung cancer, cell density and morphology were different depending on the type of cancer cells introduced. For example, when PC-9 cells, which are adenocarcinoma cells, are used, cancer cells with round nuclei and bright cytoplasms form cell aggregates with septa. However, when H520 cells, which are squamous carcinoma cells, are used, cancer cells with oval nuclei without cytoplasms proliferated to replace alveolar septa, and the pathological image was clearly different from that using PC-9 cells. Therefore, suitable cancer cells can be appropriately selected according to the type of cancer to be treated. In the above-mentioned lung cancer model, it is presumed that the cancer cells introduced into the recellularized lung engrafted in the lung and continued to proliferate, as a result of which the histopathological findings of lung cancer were reflected. Thus, even when proliferative and disease-causing cells other than cancer cells are introduced as cells for disease reproduction, a disease model that reproduces the disease can be produced in the same manner.

In addition, since a recellularized organ or tissue does not contain immunocompetent cells, any cell can be easily engrafted regardless of the type of organ or tissue. Therefore, the recellularized organ or tissue to be used in the present invention is not particularly limited, and includes heart, kidney, liver, lung, pancreas, bowel, muscle, skin, breast, esophagus, trachea, tissues thereof and the like. In the present specification, the organ includes not only the whole organ but also a part of the organ (e.g., valve of heart, etc.). The origin of the organ and the like is not particularly limited, and mammal (e.g., mouse, rat, swine, bovine, horse, goat, sheep, rabbit, kangaroo, monkey and human) can be mentioned.

Examples of the cell for disease reproduction to be used in the production method of the present invention include cancer cell, fibroblast and the like, and examples of the reproduced disease include cancer, fibrosis and the like. In the case of lung cancer model, cells of a cancer other than lung cancer are used as the cancer cells to be used in the production method of the present invention, whereby the lung cancer model can be a metastatic lung cancer model. On the other hand, when lung cancer cells are used, the lung cancer model can be a primary lung cancer model. Similarly, in the case of a cancer model other than lung cancer, the cancer model can be a metastatic cancer model or a primary cancer model depending on the type of cancer cells to be used. As the cell for disease reproduction, commercially available cells may be used, or cells newly isolated from an organ or the like (e.g., primary culture cell) may also be used. For example, cells are isolated from an organ derived from a patient who has acquired resistance to a certain pharmaceutical product as a result of treatment and used as the cell for disease reproduction, whereby therapeutic research for resistant strains becomes possible. In the case of a lung cancer model, the cancer cells to be used may be lung cancer cells or cells of a cancer other than lung cancer, with preference given to lung cancer cells. Examples of the cancer cell to be used in the production method of the present invention include cancer cells in sarcomas such as fibrosarcoma, malignant fibrous histiocytoma, liposarcoma, rhabdomyosarcoma, leiomyosarcoma, angiosarcoma, Kaposi's sarcoma, lymphangiosarcoma, synovial sarcoma, chondrosarcoma, osteosarcoma and the like, cancer types such as brain tumor, head and neck cancer, breast cancer, lung cancer, esophageal cancer, gastric cancer, duodenal cancer, appendix cancer, colorectal cancer, rectal cancer, liver cancer, pancreatic cancer, gall bladder cancer, bile duct cancer, anal cancer, kidney cancer, ureter cancer, bladder cancer, prostate cancer, penile cancer, testicular cancer, uterine cancer, ovarian cancer, vulvar cancer, vaginal cancer, skin cancer and the like, leukemia, malignant lymphoma and the like, and the like. Only one type of the above-mentioned cancer cells may be used, or two or more types thereof may be used in combination.

When cells of lung cancer are used, the cells may be those of non-small cell lung cancer (NSCLC) (e.g., adenocarcinoma (ADC), squamous carcinoma (ASC), large cell cancer (LCC)), or those of small cell lung cancer (SCLC) (e.g., small cell cancer). Examples of the adenocarcinoma cells include A549 cell, PC-9 cell, H1975 cell, HCC827 cell, A427 cell, NCI-H23 cell, NCI-H522 cell, LC174 cell, LC176 cell, LC319 cell, PC-3 cell, PC-14 cell, PC14-PE6 cell, NCI-H1373 cell, NCI-H1435 cell, NCI-H1793 cell, SK-LU-1 cell, NCI-H358 cell, NCI-H1650 cell, SW1573 cell and the like. Examples of the adenosquamous carcinoma cells include NCI-H226 cell, NCI-H596 cell, NCI-H647 cell and the like. Examples of the squamous carcinoma include H520 cell, RERF-LC-AI cell, SW-900 cell, SK-MES-1 cell, EBC-1 cell, LU61 cell, NCI-H1703 cell, NCI-H2170 cell and the like. Examples of the cells of large cell cancer include LX1 cell, FT821 cell, KTA7 cell, KTA9 cell, KTZ6 cell, PC-13 cell and the like. Examples of the cells of small cell cancer include PC-6 cell, DMS114 cell, DMS273 cell, SBC-3 cell, SBC-5 cell and the like. Among these, A549 cell, PC-9 cell, H520 cell, H1975 cell, HCC827 cell, or PC-6 cell is preferred. Only one type of the above-mentioned cancer cells may be used, or two or more types thereof may be used in combination.

Examples of the fibroblast to be used in the production method of the present invention include skin fibroblast, lung fibroblast, heart fibroblast, fibroblast of adventitia of the aorta, uterus fibroblast, villous mesenchymal fibroblast, corium fibroblast, tendon fibroblast, ligament fibroblast, synovial fibroblast, foreskin fibroblast and the like. Only one type of the above-mentioned fibroblasts may be used, or two or more types thereof may be used in combination.

Instead of the step of introducing fibroblasts, a model of fibrosis can also be produced by contacting a normal recellularized organ or tissue with a medicament that induces fibrosis. Examples of the medicament that induces fibrosis include anticancer agents such as bleomycin, gefitinib and the like, biliary disease improving drugs such as ursodeoxycholic acid and the like, shosaikoto extract, PHMG, interferon, antibiotic, carbon tetrachloride (CCl₄), dimethylnitrosoamine (DMN) and the like.

The origin of the cell for disease reproduction is not particularly limited, and mammal (e.g., mouse, rat, swine, bovine, horse, goat, sheep, rabbit, kangaroo, monkey and human) can be mentioned, with preference given to human.

The above-mentioned cells for disease reproduction may be introduced (i.e., “seeded”) into a recellularized organ or tissue (hereinafter sometimes to be referred to as “recellularized organ, etc.”) by injection at one or more positions. Furthermore, two or more types of cells (i.e., cocktail of cells or two or more divided portions) can be introduced (seeded) into a recellularized organ, etc. When two or more types of cells are introduced, for example, they may be injected at a plurality of positions of the recellularized organ, etc., or cells of different cell types may be injected into different parts of the recellularized organ, etc. Instead of or in addition to the injection, the cells for disease reproduction may be introduced by perfusion into a cannula-inserted recellularized organ, etc. A tissue (e.g., lung tissue) can also be prepared from a part of the organ (e.g., lung) thus produced.

When the cells for disease reproduction are introduced into the recellularized organ, etc. by perfusion, for example, the following steps (2-1), (2-2) or (2-3) can be performed.

(2-1) a step of perfusing a perfusion fluid containing cells for disease reproduction into a recellularized organ, etc.

(2-2) a step of perfusing a perfusion fluid containing cells for disease reproduction to the recellularized organ, etc. after perfusion of a perfusion fluid free of the cells for disease reproduction

(2-3) a step of perfusing a perfusion fluid free of the cells for disease reproduction, stopping the perfusion to allow introduction of the cells for disease reproduction into the perfusion system, and perfusing the cells together with a perfusion fluid containing a medium in the recellularized organ, etc.

The above-mentioned recellularization step may be performed a plurality of times, during which the cell type may be changed.

Examples of the perfusion fluid include, but are not particularly limited to, medium, organ preservation solution, saline, Ringer's solution, Krebs-Ringer solution and the like. Examples of the medium include, but are not particularly limited to, RPMI (Roswell Park Memorial Institute medium), MEM (Minimum Essential Medium), DMEM (Dulbecco's Modified Eagle medium), Ham's F-12 medium and the like. Examples of the organ preservation solution include, but are not particularly limited to, extracellular fluid type preservation solutions such as Celsior solution, LPD (Low potassium dextran) solution, ET-Kyoto solution and the like, intracellular solution type preservation solutions such as Euro-Collins solution, UW (University of Wisconsin) type and the like, and the like. The organ preservation solution may be an extracellular fluid type preservation solution or an intracellular solution type preservation solution. The perfusion fluid may contain as necessary additives suitable for maintaining cells and the like such as plasma, serum, amino acid and the like.

The contact time between the perfusion fluid and the recellularized organ, etc. is preferably not less than 5 min, more preferably not less than 20 min, from the aspect of spreading the perfusion fluid to the entire recellularized organ, etc. and sufficient diffusion thereof. The upper limit of the contact time between the perfusion fluid and the recellularized organ, etc. can be appropriately determined according to, for example, the type of the recellularized organ, etc., the degree of adhesion of the cells for disease reproduction, and the like.

The flow rate of the perfusion fluid may be a flow rate generally used in perfusion of the recellularized organ, etc. It is preferably not less than 0.01 mL/min, more preferably not less than 0.1 mL/min. The flow rate of the perfusion fluid is preferably not more than 100 mL/min, more preferably not more than 20 mL/min. The temperature of the perfusion fluid during contact between the recellularized organ, etc. and the perfusion fluid is not particularly limited. For example, 4-40° C. is preferable, and 20-38° C. is more preferable.

The number of the cells for disease reproduction to be used in the present invention can be appropriately determined according to the size and weight of the recellularized organ, etc., the type and the like of the cell for disease reproduction, and the like. For example, it is preferable to seed at least about 1,000 (e.g., not less than 10,000, not less than 100,000, not less than 1,000,000, not less than 10,000,000 or not less than 100,000,000) cells for disease reproduction in the case of a recellularized organ, etc. Alternatively, it is preferable to seed about 1,000-about 10,000,000 cells for disease reproduction per 1 mg of a recellularized organ, etc.

The origin of the recellularized organ, etc. is not particularly limited, and mammal (e.g., mouse, rat, swine, bovine, horse, goat, sheep, rabbit, kangaroo, monkey and human) can be mentioned.

The recellularized organ, etc. can be produced by a method known per se and can be produced by, for example, recellularizing a decellularized organ or tissue (hereinafter sometimes to be referred to as “decellularized organ, etc.”).

Specifically, the methods described in Thomas H. et al., Science, 329(5991): 538-41 (2010), Fecher D. et al., PLoS One, 11(8): e0160282 (2016) and the like can be used in the case of lung, the methods described in Bao J. et al., Cell Transplant, 20(5): 753-766 (2011), Barakat O. et al., J. Surg Res, 173(1): e11-e25 (2012), Soto-Gutierrez A. et al., Tissue Eng Part C Methods, 17(6): 677-686 (2011), Uygun B. E. et al., Nat Med, 16(7): 814-820 (2010) and the like can be used in the case of liver, the methods described in WO 2010/120539, WO 2012/031162 and the like can be used in the case of heart, the methods described in Mireia Caralt et al., Am J Transplant, 15(1):64-75 (2015) and the like can be used in the case of kidney, and the methods described in Hwang J. et al., Acta Biomater, 53: 268-278 (2017), White L. J. et al., Acta Biomater, 50: 207-219 (2017) and the like can be used in the case of bladder.

More specifically, as a method for recellularizing lung or lung tissue, for example, a method including a step of introducing a cell suspension containing epithelial cells into the airway compartment by perfusion or injection, and a step of seeding the endothelial cells into the lung by perfusion or injection can be performed. At this time, air may be sent to the decellularized lung (also referred to as decellularized lung) during the introduction of the endothelial cell population, thereby allowing the spread of the seeded endothelial cells. From the aspect of maturation of regenerated blood vessels, it is preferable to introduce mesenchymal stem cells at the time of or before or after introduction of endothelial cells.

In the present specification, the “recellularization” refers to introducing cells into a decellularized organ, etc. and engrafting the introduced cells in a part or entirety of the decellularized organ, etc. (hereinafter sometimes to be referred to as “cell for recellularization”). In the present specification, moreover, the “decellularization” means removing cell components from living organ or tissue, and the “decellularized organ or tissue” means a scaffold having an extracellular matrix as a main component and having a three-dimensional structure in which the cell components are removed from the living organ or tissue. In decellularization, the cell components may be completely removed, but complete removal of the cell components is not necessarily required, and the case of a decrease in cell components compared to the organ or tissue before decellularization is also called decellularization. In addition, sulfated glycosaminoglycan (GAG), which is one of the extracellular matrices, may or may not remain in the decellularized organ, etc. used in the production method of the present invention.

The method for introducing cells for recellularization by perfusion or injection, the contact time between the perfusion fluid and the decellularized organ, and the flow rate and temperature of the perfusion fluid may be the same as those for introducing the above-mentioned cells for disease reproduction, and similar conditions can be used. In this case, the “cells for disease reproduction” is to be read as “cells for recellularization” and “recellularized organ, etc.” is to be read as “decellularized organ, etc.”

As the perfusion fluid, the same perfusion fluid as in the above-mentioned case of introducing the cells for disease reproduction into a recellularized organ, etc. can be used, and the perfusion fluid may contain the same additives and the like as those described above.

The number of the cells for recellularization to be used in the present invention can be appropriately determined depending on both the size and weight of the decellularized organ, etc., the type and the like of the cell for recellularization, and the like. For example, it is preferable to seed at least about 1,000 (e.g., not less than 10,000, not less than 100,000, not less than 1,000,000, not less than 10,000,000 or not less than 100,000,000) cells for recellularization in the case of a decellularized organ, etc. Alternatively, it is preferable to seed about 1,000-about 10,000,000 cells per 1 mg of an organ and the like (wet weight, i.e., weight before decellularization).

Examples of the epithelial cell to be used for recellularization include alveolar epithelial cell (e.g., TYPE I alveolar epithelial cell, TYPE II alveolar epithelial cell), Clara cell, goblet cell and the like, with preference given to alveolar epithelial cell.

Examples of the endothelial cell to be used for recellularization include blood endothelial cell, bone marrow endothelial cell, circulating endothelial cell, aortic endothelial cell, brain microvascular endothelial cell, skin microvascular endothelial cell, intestinal microvascular endothelial cell, lung microvascular endothelial cell, microvascular endothelial cell, liver sinusoidal endothelial cell, saphenous vein endothelial cell, umbilical vein endothelial cell, lymphatic endothelial cell, microvascular endothelial cell, microvascular endothelial cell, pulmonary artery endothelial cell, retina capillary vessel endothelial cell, retina microvascular endothelial cell, vascular endothelial cell, cord blood endothelial cell, liver sinusoidal endothelial cell, endothelial cell colony formation unit (CFU-EC), circulating angiogenic cell (CAC), circulating endothelial progenitor cell (CEP), endothelial colony forming cell (ECFC), low proliferative potential endothelial ECFC (LPP-ECFC), high proliferative potential ECFC (HPP-ECFC) and the like, preferably lung microvascular endothelial cell (LMVEC).

Examples of the mesenchymal stem cell to be used for recellularization include stem cells derived from bone marrow fluid, adipose tissue, placenta tissue, umbilical cord tissue, dental pulp and the like. In view of low invasiveness during collection, adipose-derived mesenchymal stem cell (ADSC) is preferred.

The origin of the cell for recellularization is not particularly limited, and mammals same as those as the origin of the cell for disease reproduction can be mentioned, with preference given to human.

The decellularized organ, etc. can be produced by a method known per se (e.g., the method described in WO 2010/120539, the method described in WO 2012/031162, the method described in Fecher D. et al., PLoS One, 11(8): e0160282 (2016), etc.). For example, it can be produced by contacting an organ or tissue isolated from a living organism with a decellularization solution containing a surfactant (e.g., sodium dodecyl sulfate acid (SDS), sodium deoxycholate (SDC), CHAPS, Triton X-100, etc.) by perfusion, and the like. The decellularized organ, etc. is preferably washed with a solution containing a nuclease enzyme to decompose nucleic acid substances remaining in the organ and the like.

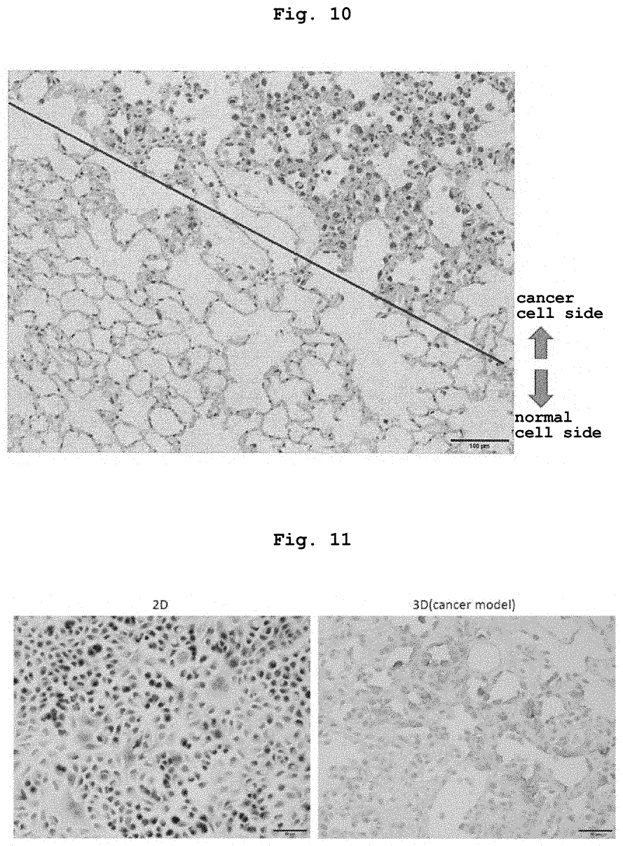

As shown in Examples described below, progression of cancer cells during the process of the production method of the present invention can be observed (FIG. 10). Thus, the production method of the present invention may also be useful for the study of progression of cancer. Therefore, in another embodiment, a method for observing progression of a cell for disease reproduction is provided which includes a step of introducing a cell for disease reproduction (preferably cancer cell) into a recellularized organ, etc. The observation method can also be performed real-time by live imaging and the like. The type, seeding method and the like of the cell for disease reproduction to be used are as mentioned above.

### 2. Disease Model

The present invention also provides a disease model produced by the production method of the present invention (hereinafter sometimes to be referred to as “the disease model of the present invention”), preferably a lung disease model (e.g., lung cancer model). As mentioned above, the lung cancer model of the present invention can reflect the histopathological findings of naturally-occurring lung cancer. That is, in one embodiment, the lung disease model of the present invention has a nodule in the site of cancer cell introduction, has a grand duct-like structure, and has mucus in the cells. Therefore, the disease model of the present invention which can reproduce a disease is suitable for screening for an agent for treating or preventing a disease. In addition, for example, a more physiological mechanical stress such as perfusion to pulmonary blood vessels, addition of respiratory movement, or the like can be added to the lung disease model in a bioreactor. Therefore, the disease model of the present invention is also useful in elucidating the biological mechanism of a disease, including elucidation of mechanobiology.

### 3. Method for Screening for Agent for Treating or Preventing Disease

The present invention provides a method for screening for a medicament useful for treatment or prophylaxis of a disease (hereinafter to be also referred to as “the screening method of the present invention”). The screening method of the present invention includes, for example, (1) a step of contacting the disease model of the present invention with a test substance, and (2) a step of selecting the test substance as a candidate substance for treating and/or preventing a disease when the contact with the test substance decreases the number of cells for disease reproduction, or decreases the proliferation rate of the cell, compared with the disease model before contact with the test substance or a disease model without contact with the test substance or a disease model contacted with a control substance known to be ineffective for the treatment or prophylaxis of the disease. The measurement of the number of the cells for disease reproduction and proliferation rate can be performed by methods known per se, for example, a method of staining a tissue section to measure the number of cells, a method of performing image analysis (e.g., analysis using ImageJ, etc.), a method of live imaging, and the like.

Examples of the disease to be the target of the above-mentioned therapeutic or prophylactic agent include cancer (e.g., lung cancer), fibrosis (e.g., lung fibrosis) and the like. Examples of the aforementioned cancer include sarcomas such as fibrosarcoma, malignant fibrous histiocytoma, liposarcoma, rhabdomyosarcoma, leiomyosarcoma, angiosarcoma, Kaposi's sarcoma, lymphangiosarcoma, synovial sarcoma, chondrosarcoma, osteosarcoma and the like, cancer types such as brain tumor, head and neck cancer, breast cancer, lung cancer, esophageal cancer, gastric cancer, duodenal cancer, appendix cancer, colorectal cancer, rectal cancer, liver cancer, pancreatic cancer, gall bladder cancer, bile duct cancer, anal cancer, kidney cancer, ureter cancer, bladder cancer, prostate cancer, penile cancer, testicular tumor, uterine cancer, ovarian cancer, vulvar cancer, vaginal cancer, skin cancer and the like, leukemia malignant lymphoma and the like, among which lung cancer is preferred. Examples of the lung cancer include non-small cell lung cancer (e.g., adenocarcinoma (ADC), squamous carcinoma (ASC), large cell cancer (LCC)), small cell lung cancer (SCLC) (e.g., small cell cancer) and the like. Examples of the aforementioned fibrosis include lung fibrosis, liver fibrosis, pancreatic fibrosis, kidney fibrosis, heart fibrosis, myelofibrosis, skin fibrosis and the like.

In the present specification, the test substance includes, for example, biological samples (e.g., blood, serum, plasma, etc.) containing an immunocyte (e.g., dendritic cell, lymphocyte (e.g., T cell, B cell, natural killer cell, etc.), macrophage, etc.) or a blood cell (e.g., erythrocyte, leukocyte (e.g., neutrophil, eosinophil, basophil, lymphocyte, monocyte, etc.), platelet etc.) or variants thereof, cell extracts, cell culture supernatants, microbial fermentation products, extracts from marine organisms, plant extracts, purified or crude proteins, peptides, non-peptide compounds, synthetic low-molecular-weight compounds, and natural compounds.

In the present specification, the test substances can also be obtained using any of the many approaches in the combinatorial library methods known in the art including (1) biological libraries, (2) synthetic library methods using deconvolution, (3) “(one-bead one-compound)” library method, and (4) synthetic library methods using affinity chromatography selection. The biological library method using affinity chromatography selection is limited to peptide library, but the other four approaches are applicable to low-molecular-weight compound libraries of peptides, non-peptide oligomers, or compounds (Lam (1997) Anticancer Drug Des. 12:145-67). Examples of a method for synthesizing a molecular library can be found in the art (DeWitt et al. (1993) Proc. Natl. Acad. Sci. USA 90:6909-13; Erb et al. (1994) Proc. Natl. Acad. Sci. USA 91:11422-6; Zuckermann et al. (1994) J. Med. Chem. 37:2678-85; Cho et al. (1993) Science 261:1303-5; Carell et al. (1994) Angew. Chem. Int. Ed. Engl. 33:2059; Carell et al. (1994) Angew. Chem. Int. Ed. Engl. 33:2061; Gallop et al. (1994) J. Med. Chem. 37:1233-51). Compound libraries can be produced as a solution (see Houghten (1992) Bio/Techniques 13:412-21) or bead (Lam (1991) Nature 354:82-4), chip (Fodor (1993) Nature 364:555-6), bacterium (U.S. Pat. No. 5,223,409), spore (U.S. Pat. Nos. 5,571,698, 5,403,484, and 5,223,409), plasmid (Cull et al. (1992) Proc. Natl. Acad. Sci. USA 89:1865-9) or phage (Scott and Smith (1990) Science 249:386-90; Devlin (1990) Science 249:404-6; Cwirla et al. (1990) Proc. Natl. Acad. Sci. USA 87:6378-82; Felici (1991) J. Mol. Biol. 222:301-10; US-B-2002103360).

### 4. Evaluation Method of Side Effect of Agent for Treating or Preventing Disease

The present invention provides a method for evaluating a side effect of a test substance (hereinafter to be also referred to as “the evaluation method of the present invention”). The evaluation method of the present invention includes, for example, (1) a step of contacting the disease model of the present invention with a test substance, and (2) a step of evaluating the level of damage to the disease model due to the contact with the test substance. The evaluation in the above-mentioned step (2) can be performed by, for example, measuring the number of remaining normal cells before and after contact with the test substance, and calculating a decrease in the normal cells due to the contact with the test substance. As a disease model to be a comparison target, a disease model without contact with a test substance, or a disease model contacted with a control substance with a known side effect or known to have no side effect may also be used. Alternatively, the degree of side effects of two or more kinds of test substances can also be evaluated using a disease model contacted with a different kind of test substance as a comparison target. The number of normal cells can be measured by the same method as the measurement of the number of cells for disease reproduction in the above-mentioned 3.

When using a lung disease model, the side effects of a test substance can also be evaluated by measuring the oxygen exchange rate of the lung instead of measuring the number of remaining normal cells. The measurement of the oxygen exchange rate of the lung can be performed by, for example, injecting a mixture of artificial erythrocytes and deoxygenized PBS from the pulmonary artery while ventilating from the trachea of the regenerate lung, and comparing the oxygen partial pressures between before injection and after injection recovered from the pulmonary vein.

The test substance to be used in the evaluation method of the present invention may be a therapeutic or prophylactic agent for a disease whose therapeutic or prophylactic effect is already known, or a test substance whose effect is unidentified, for example, the test substances described in the above-mentioned 3, or a candidate substance obtained by the screening method of the present invention.

While the present invention is further described in the following by way of Examples, the present invention is not limited thereby.

### Example

### Example 1 Production of Lung Cancer Model

### Method

### 1. Collection of Rat Lung

Lungs were collected from young adult (3 months old) male Fisher 344 rats (Charles River, Wilmington, Mass.). All animal experiments were conducted with the approval of the Institutional Animal Care and Use Committee of Nagasaki University, and in accordance with the animal experiment guidelines of Nagasaki University. Rats were euthanized by intraperitoneal injection of pentobarbital sodium (Sigma, 140 mg/kg) and heparin (250 U/kg). The diaphragm was punctured and the thorax was amputated to expose the lung. Lungs were perfused via the right ventricle with PBS containing 50 U/ml heparin (Sigma) and 1 μg/ml sodium nitroprusside (SNP, Fluka). After completion of the perfusion, the heart, lungs and trachea were dissected and removed all together. 18 G and 14 G catheters were respectively cannulated into the pulmonary artery, pulmonary vein and trachea.

### 2. Production of Lung Cancer Model Lung

### 2-1. Decellularization of Rat Lung

The collected and cannulated rat lung was placed in a bioreactor and the pulmonary artery was connected. After perfusing 100 ml of PBS+, 425 ml of 0.0035% Triton PBS+ solution was perfused. Then, 250 ml of Benz buffer (mixture of Tris-HCl, MgCl₂, BSA and Milli-Q adjusted to pH 8) was perfused. Furthermore, 150 ml of PBS− and 1M NaCl solution was perfused, and then rinsed with 250 ml of PBS−. Then, 425 ml each of SDS solution was perfused in the order of 0.01%, 0.05%, and 0.1%. After rinsing with 425 ml of PBS−, 100 ml of Triton 0.5%+EDTA solution was perfused. After 2000 ml of PBS− was flown, 500 ml of PBS− mixed with Penicillin+Streptomycin, Amphotericin B, and Gentamycin was finally flown. The lung was immersed in this solution for storage. All perfusions were performed with gravity from a height of 30 cm.

### 2-2. Recellularization of Decellularized Lung

Fischer 344 rats at 3-4 weeks were euthanized by intraperitoneal injection of pentobarbital sodium (Sigma, 140 mg/kg) and heparin (250 U/kg). The trachea was secured and cannulated with a 14 G surflo needle. The diaphragm was punctured and the thorax was amputated to expose the lung. Lungs were perfused via the right ventricle with PBS containing U/ml heparin (Sigma) and 1 μg/ml sodium nitroprusside (SNP, Fluka). After completion of the perfusion, the heart, lung and trachea were dissected and removed all together. The trachea was rinsed with cooled PBS−, 1.5 ml of a solution of DMEM+2.5% HEPES+elastase (4.5 U/ml)+DNase I (0.02 mg/ml) (Solution A) was injected, and 0.5-1.0 ml of a 1% low-melting point agarose was immediately injected and cooled. The trachea was ligated, the of surflo-needle was decannulated, the heart was resected and the lungs were placed in a Falcon tube containing Solution A. Lungs of a total of 3-4 rats were treated in the same manner and shaken at 37° C., 100 beats/min for 45 min. The trachea and ¼ on the central side were resected in a clean bench, and peripheral ¾ lungs were collected and chopped into small pieces with scissors or a scalpel. New Solution A and chopped lung tissue were placed in a new Falcon tube and shaken at 37° C. for 15 min at 100 times/min. The elastase reaction was quenched by adding DMEM+2.5% HEPES+50% FBS, and the mixture was filtered through 100 μm and 70 μm nylon mesh and centrifuged at 300×g. The supernatant was aspirated and the pellet was suspended in DMEM/F12+10% FBS containing antibiotic and antifungal agent to complete the production of alveolar epithelial suspension.

The trachea of the decellularized rat lung was connected to the bioreactor in a clean bench, and the extracted suspension of the alveolar epithelium of the rat was flown into the trachea from a height of 60 cm with gravity. The bioreactor was then allowed to stand overnight in the CO₂ incubator.

The next day, the bioreactor was moved into the clean bench again, and 120 ml of a 1:1 mixture of DMEM/F12 and EGM-2 was flown into the pulmonary artery with gravity. A suspension of adipose stem cells (ADSC) extracted from rats of the same strain as rat lung microvascular epithelial cells (RLMVEC) was produced and perfused by gravity from the pulmonary artery and pulmonary vein. 3.0-4.0×10⁷ RLMVEC were used and 6.0-8.0×10⁵ ADSC were used. After perfusion, the bioreactor was allowed to stand in a CO₂ incubator for 90 min. Thereafter, the pulmonary artery was perfused with a pump at 1 ml/min, and simultaneously, DMEM/F12 was perfused from the trachea with a pump so as to repeat inflow and outflow at 5-10 ml per minute.

Thereafter, the pulmonary artery pump was increased by 1 ml/min every day and perfusion was performed at 4 ml/min at maximum.

### 2-3. Seeding of Cancer Cell

### (1) Seeding of A549 Cell

The day after perfusion of the RLMVEC+ADSC suspension, a suspension of A549 was produced. The suspension was adjusted to 1-2×10⁶ per 40-50 μl. The bioreactor was moved into a clean bench and 40-50 μl of the suspension of A549 was locally injected into any site of rat regenerated lung with an insulin injection syringe. The bioreactor was then allowed to stand in a CO₂ incubator for 60 min. To prevent cancer cells from being seeded in other sites, all the medium in the bioreactor was once aspirated, then a new medium was added, and perfusion of the pulmonary artery and trachea was resumed in the CO₂ incubator.

### (2) Seeding of PC-9 Cell

A suspension was prepared by the same method as in (1) and locally injected by the same method.

### (3) Seeding of H520 Cell

A suspension was prepared by the same method as in (1) and locally injected by the same method.

### Example 2 Histological Analysis

### Method

### Hematoxylin-Eosin Staining

Samples (decellularized lung, recellularized lung or recellularized lung injected with cancer cell) were fixed for 4 hr in 10% formalin or 4% paraformaldehyde, dehydrated, embedded in paraffin, 5 μm sections were produced, and Hematoxylin-Eosin (H&E) staining was performed.

### Periodic Acid-Schiff Staining

Samples (decellularized lung, recellularized lung or recellularized lung injected with cancer cell) were fixed for 4 hr in 10% formalin, dehydrated, embedded in paraffin, 5 μm sections were produced. Thereafter, the sections were deparaffinized and xylene was removed, and the sections were washed with water for several seconds, then immersed in 0.5% periodic acid solution for 10 min, washed with running water for 5 min, then immersed in distilled water for 2 min, and further immersed in Schiff's reagent for 15 min. Then, the sections were immersed in a sulfite solution for 2 min, 3 times, and then washed with running water for 5 min. They were immersed in Meyer's Hematoxylin Solution for 2 min, washed with running water for 1 min, color was developed with warm water or aqueous ammonia water at 60° C. for 10 min, and then dehydrated, cleared, and sealed to complete Periodic Acid-Schiff staining.

### Results

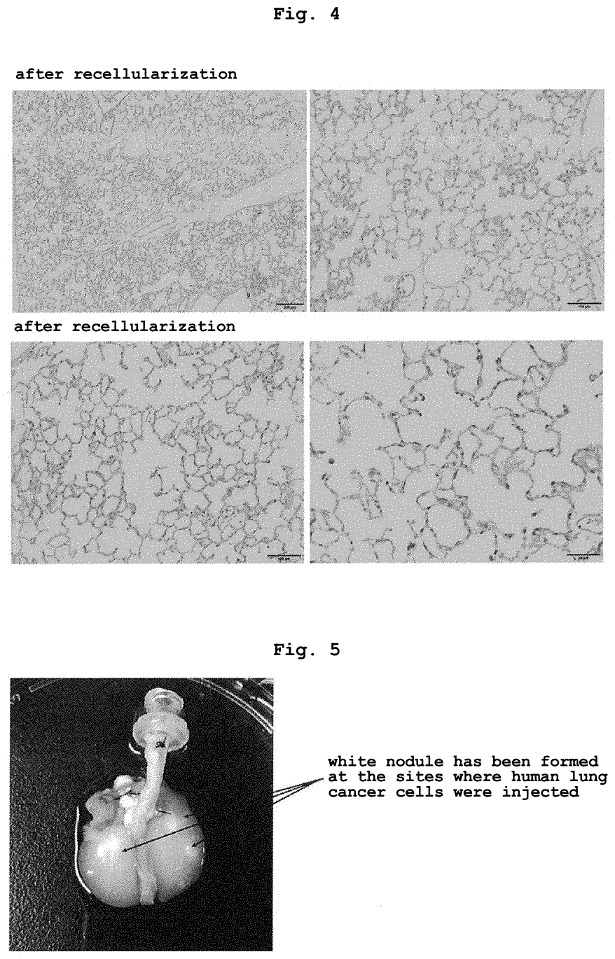

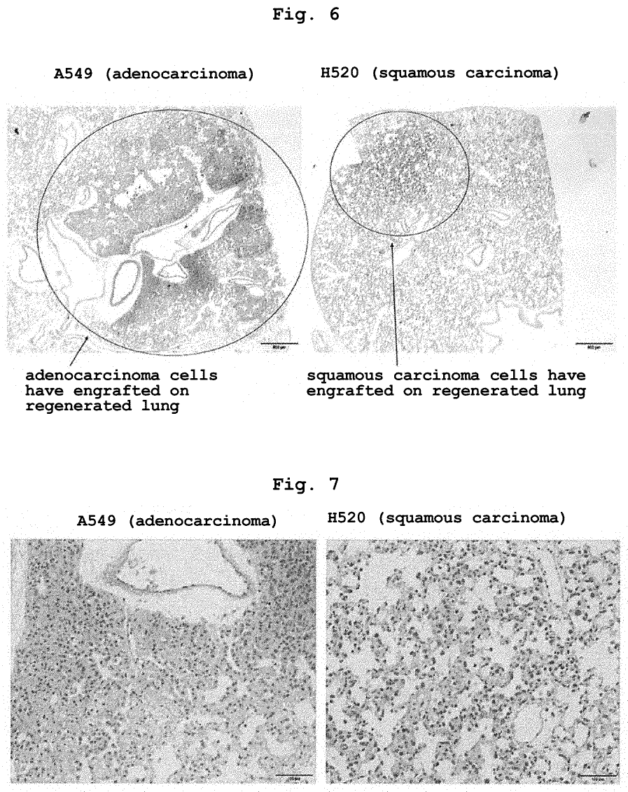

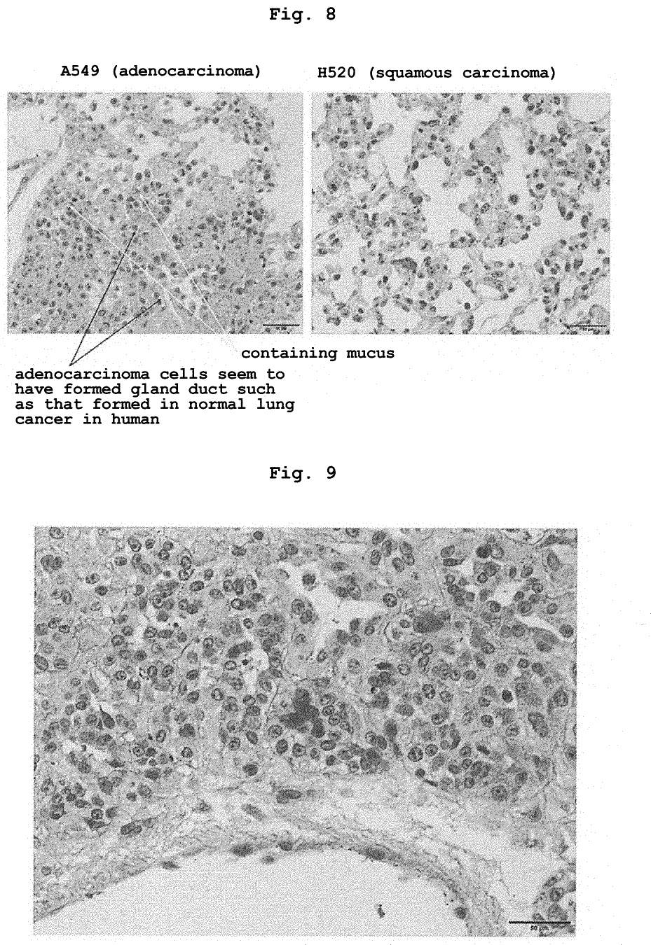

The alveolar epithelial cells and vascular endothelial cells that were perfused during recellularization were engrafted to reproduce the normal alveolar structure while maintaining the alveolar structure of the decellularized scaffold (FIG. 4). In addition, both adenocarcinoma cells and squamous carcinoma cells were engrafted on the recellularized lung (FIG. 6). When PC-9 cells, which are human lung cancer cells, were used, white nodules were observed at the sites where the cells were injected (FIG. 5). In the lungs injected with adenocarcinoma cells as lung cancer cells, gland duct-like structures were formed, mucus was contained in the cells (FIG. 8), and mucus that turns reddish purple by Periodic Acid-Schiff staining was also observed in the gland duct-like structures and cells (FIG. 9). When PC-9 cell is used, cancer cells with round nuclei and bright cytoplasms form cell aggregates with septa. However, when H520 cells, which are squamous carcinoma cells, are used, cancer cells with oval nuclei without cytoplasms proliferated to replace alveolar septa, and the pathological image is clearly different (FIG. 7). This suggests that the mode of infiltration differs depending on the type of cell used and character thereof. Therefore, the lung cancer model produced by the production method of the present invention was shown to reflect the histopathological findings of naturally-occurring lung cancer, that is, reproduce naturally-occurring lung cancer. Furthermore, the progression of cancer cells could also be observed (FIG. 10).

### Example 3 Histological Analysis (Immunostaining)

MUC-1 is expressed in many solid cancer cells. In particular, the C-terminal side is considered to be involved in the proliferation of cancer cells, promotion of infiltration ability, suppression of apoptosis, and the like through interaction with multiple molecules related to signal transduction. In general, the expression level of MUC-1 increases in cancer cells. MUC-1 is expressed with polarity on the cell surface in normal epithelial cells, but the polarity is considered to be lost in cancer cells (depolarized expression pattern). Using MUC-1 as an index, whether or not the lung cancer model produced by the production method of the present invention reproduces naturally-occurring lung cancer was verified.

### Method

In immunostained samples (two-dimensionally cultured lung cancer cell line (2D), recellularized lung (3D) injected with cancer cells), 2D was fixed with 4% paraformaldehyde (PFA) for 10 min and 3D was fixed with 4% PFA for 24 hr. 2D was directly used for staining, or when there was some time before use, immersed in PBS(−), stored at 4° C., and used within 1 week. 3D was embedded in paraffin, 5 μm sections were prepared, deparaffinized prior to staining, and then antigen retrieval by heat treatment and endogenous peroxidase blocking were performed. After blocking with PBS(−)+5% normal goat serum in both 2D and 3D, the primary antibody (MUC1-C (D5K9I, 1:400, Cell Signaling Technology, #16564)) was reacted at 4° C. overnight. Successively, the secondary antibody was reacted at room temperature for 1 hr, then color was developed with 3,3′-diaminobenzidine (DAB) and nuclear staining was performed with Hematoxylin.

### Results

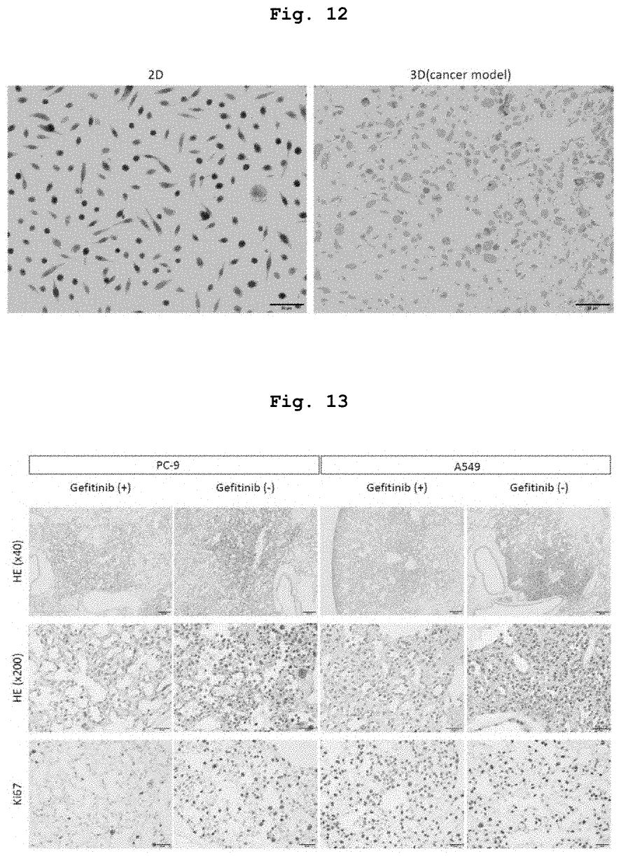

When either A549 cells or PC-9 cells were used as the injected cancer cells, MUC-1 was hardly expressed in 2D (FIG. 11 left Figure, FIG. 12 left Figure), but in 3D, the expression level of MUC-1 increased (FIG. 11 right Figure, FIG. 12 right Figure). That is, the lung cancer model produced by the production method of the present invention is considered to reproduce naturally-occurring lung cancer more than the two-dimensionally cultured cancer cell line.

### Example 4 Verification of Responsiveness to Anticancer Agent

Finally, whether or not the lung cancer model produced by the production method of the present invention has the same responsiveness to anticancer agents as that of naturally-occurring lung cancer was verified.

### Method

### Administration of Gefitinib

1.4 μl of a gefitinib solution obtained by suspending gefitinib at 100 μg/ml in dimethyl sulfoxide (DMSO), followed by filter sterilization, was added to a total of 140 ml of a medium obtained by mixing DMEM/F-12 medium and EGM-2 at 1:1 to prepare a medium containing 1 μM gofitinib. For ventilation, 0.6 μl of a gefitinib solution was added to 60 ml of DMEM/F-12 medium and adjusted to 1 μM. After culturing for 3 days a sample of recellularized lung seeded by locally injecting A549 and PC-9, all the medium was aspirated, a medium containing 1 μM gefitinib prepared as described above was newly added to the bioreactor and a bottle for ventilation, and the sample was cultured for 48 hr. In addition, as a control, a group to which the same amount of DMSO was added without adding gefitinib was also prepared and cultured for 48 hr in the same manner.

Immunostained samples (recellularized lung (3D) injected with cancer cells) were fixed with 4% PFA for 24 hr. 3D was embedded in paraffin, and 5 μm sections were produced. After deparaffinizing, antigen retrieval by heat treatment and endogenous peroxidase blocking were performed. Successively, blocking with PBS(−)+5% normal goat serum was performed, the primary antibody (Ki67 (SP6, 1:1000, Abcam, ab16667)) was reacted at 4° C. overnight. Successively, the secondary antibody was reacted at room temperature for 1 hr, then color was developed with 3,3′-diaminobenzidine (DAB) and nuclear staining was performed with Hematoxylin.

### Hematoxylin-Eosin Staining

Samples (recellularized lung injected with cancer cells) was fixed for 4 hr in 10% formalin, dehydrated, embedded in paraffin, 5 μm sections were produced, and stained with Hematoxylin-Eosin (H&E).

### Calculation of Percentage of Ki67 Positive Cells

The recellularized lungs seeded with A549 and PC-9 were divided into a gefitinib administration group and a control group, and a total of 12 samples (each n=3) were prepared. After staining with Ki67 by immunostaining as described above, obvious cancerous sites were identified, and 10 fields each were randomly photographed at ×400 with an optical microscope. Using an image analysis software ImageJ, the total number of cells and the number of positive cells in each field were counted, and the positive cell rate was calculated. The statistically significant difference in the Ki67-positive cell rate of each group was calculated by t-test, and a p value <0.05 was taken as statistically significant.

### Results

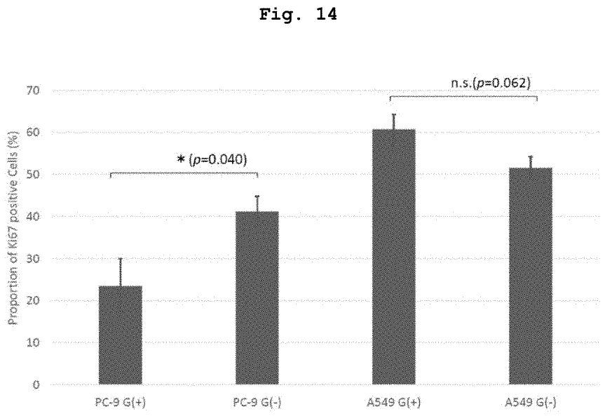

When A549 cells (EGFR is wild-type) were used as the injected cancer cells, no significant difference was found in the expression of a cell proliferation marker Ki67 by the presence or absence of gefitinib administration (however, the number of Ki67-positive cells tended to decrease by gefitinib administration) (FIG. 13, FIG. 14). On the other hand, when PC-9 cells (EGFR has a mutation) were used as the injected cancer cells, the number of Ki67-positive cells significantly decreased (that is, proliferation was suppressed) by gefitinib administration (FIG. 13, FIG. 14). At the laboratory level, gefitinib has been reported to be also effective against EGFR with normal structure (Int J Cancer. 2001 Dec. 15; 94(6):774-82, Clin Cancer Res. 2001 October; 7(10):2958-70). In actual clinical practice, it has been reported that gefitinib particularly shows a tumor-reducing effect when the EGFR gene in tumor cells is associated with a special type of mutation (N Engl J Med. May 20; 350(21):2129-39, Science. 2004 Jun. 4; 304(5676):1497-500). Therefore, the results of this Example are consistent with known reports, and the lung cancer model produced by the production method of the present invention is considered to have the same responsiveness to anticancer agents as that of naturally-occurring lung cancer.

### INDUSTRIAL APPLICABILITY

According to the present invention, a disease model having a three-dimensional structure can be produced. The disease model thus produced can be used for elucidation of the biological mechanism of a disease and more accurate prediction of the effect of an agent for treating or preventing a disease.

This application is based on a patent application No. 2019-014778 filed in Japan (filing date: Jan. 30, 2019), the contents of which are incorporated in full herein.

1. A method for producing a disease model, comprising a step of introducing a cancer cell or fibroblast into a recellularized organ or tissue.

2. The method according to claim 1, wherein the organ or tissue is a lung or lung tissue.

3. The method according to claim 1, wherein the cancer cell is a lung cancer cell.

4. The method according to claim 1, wherein the cancer cells are one or more types of cells selected from the group consisting of A549 cell, PC-9 cell, H520 cell, H1975 cell, HCC827 cell and PC-6 cell.

5. The method according to claim 2, wherein the recellularized lung or lung tissue is a lung or lung tissue produced by introducing an epithelial cell and an endothelial cell into a decellularized lung or lung tissue.

6. A disease model produced by the method according to claim 1.

7. The disease model according to claim 6, wherein the disease model is a lung disease model.

8. A method for screening for an agent for treating or preventing a disease, comprising
(1) a step of contacting the disease model according to claim 6 with a test substance, and
(2) a step of selecting the test substance as a candidate substance for treating or preventing a disease when the contact with the test substance decreases the number of cancer cells or fibroblasts, or decreases the proliferation rate of the cell, compared with the disease model before contact with the test substance or a disease model without contact with the test substance.

9. The method according to claim 8, wherein the disease is a lung disease.

10. A method for evaluating a side effect of a test substance, comprising
(1) a step of contacting the disease model according to claim 6 with the test substance, and
(2) a step of evaluating the level of damage to the disease model due to the contact with the test substance.

11. The method according to claim 2, wherein the cancer cell is a lung cancer cell.

12. The method according to claim 11, wherein the recellularized lung or lung tissue is a lung or lung tissue produced by introducing an epithelial cell and an endothelial cell into a decellularized lung or lung tissue.

13. A disease model produced by the method according to claim 2.

14. The disease model according to claim 13, wherein the disease model is a lung disease model.

15. A disease model produced by the method according to claim 3.

16. The disease model according to claim 15, wherein the disease model is a lung disease model.

17. A disease model produced by the method according to claim 5.

18. The disease model according to claim 17, wherein the disease model is a lung disease model.

19. A disease model produced by the method according to claim 11.

20. The disease model according to claim 19, wherein the disease model is a lung disease model.
